(19) United States
(12) Patent Application Publication
(10) Pub. No.: US 2008/0195050 A1
(43) Pub. Date: Aug. 14, 2008

(54) Catheter and medical tubing securement device

(75) Inventors: Aaron M. Dickert, Grand Ledge, MI (US); Gregory A. Sloan, Ann Arbor, MI (US)

Correspondence Address: Christopher J. Fildes;Fildes & Outland, P.C., Suite 2, 20916 Mack Avenue, Grosse Pointe Woods, MI, 48236, US

(73) Assignee: Tri-State Hospital Supply Corporation

(21) Appl. No.: 11/705,204

(22) Filed: Feb. 12, 2007

(51) Int. Cl. A61F 13/02

(52) U.S. Cl. 604/180

(57) ABSTRACT

A catheter and medical tubing securement device includes a layer member having an adhesive coated side and an opposite non-adhesive side. The adhesive coated side is adherable to a patient's skin. An adjacent tab is removably connected to the layer member. A user may manipulate the layer member by grasping the tab, and the tab may be removed from the layer member by tearing the tab from the layer member.

### TECHNICAL FIELD

This invention relates to catheter and medical tubing securement devices, and more particularly to catheter and medical tubing securement devices that are easily manipulated.

### BACKGROUND OF THE INVENTION

It is known that a health care provider performing catheter insertion and securement as well as the connection of medical tubing to a catheter will typically wear gloves during the procedure. Certain catheter and medical tubing securement devices have adhesive sides that are ultimately mounted on a catheter and/or medical tubing and a patient's skin. In order to manipulate these securement devices, a health care provider must grasp the securement devices with his/her gloved fingers. In an ideal situation, the health care provider's gloved fingers make minimal contact with the adhesive, and the health care provider is able to apply the securement device without the adhesive becoming stuck to the glove. More commonly, in a less than ideal situation, when a health care provider grasps a securement device, the adhesive becomes stuck to the health care provider's gloved fingers and it is difficult to separate the securement device from the glove when mounting the securement device on a patient. Also, it is difficult for a health care provider to properly position and align the securement device when it is stuck to his/her gloved fingers.

### SUMMARY OF THE INVENTION

The present invention provides a catheter and medical tubing securement device that will not become undesirably stuck to a user's fingers during application. The catheter and medical tubing device includes a grip tab that a health care provider can hold while manipulating and positioning the securement device, preventing the health care provider's gloved fingers from coming in contact with adhesive on the securement device. Once the securement device has been mounted on a patient, the tab may be removed from the securement device and discarded.

More particularly, a catheter and medical tubing securement device in accordance with the invention includes a layer member having an adhesive coated side and an opposite non-adhesive side. The adhesive coated side is adherable to a patient's skin. An adjacent tab is removably connected to the layer member by frangible lands. A user may manipulate the layer member by grasping the tab, and the tab may be removed from the layer member by tearing the frangible lands.

In a specific embodiment, the frangible lands may define a perforated boundary line, a scored line, or similar. The tab may have opposite non-adhesive sides. Alternatively, the tab may have an adhesive coated side and an opposite non-adhesive side. The adhesive coated side of the tab may correspond with the adhesive coated side of the layer member. A release liner may be disposed on the adhesive coated side of the tab. The release liner generally may correspond in shape with the tab. Another release liner having a tackless side may be removably disposed on the layer member adhesive coated side.

The tab may be integral with the layer member. The layer member and/or the tab may be made of a foam material or similar.

In another embodiment of the invention, a catheter and medical tubing securement device includes a layer member having an adhesive coated side and an opposite non-adhesive side. The adhesive coated side is adherable to a patient's skin. The layer member includes a tab portion and an adjacent securing portion. A boundary between the tab portion and the securing portion is defined by perforations extending through the layer member. The layer member may be grasped by the tab portion, and the tab portion may be removed from the securing portion by tearing the tab portion from the securing portion along the boundary.

Optionally, a release liner may be removably disposed on the layer member adhesive coated side. The release liner generally may extend beyond an outer edge of the layer member. The layer member may be made of a foam material or similar.

These and other features and advantages of the invention will be more fully understood from the following detailed description of the invention taken together with the accompanying drawings.

### BRIEF DESCRIPTION OF THE DRAWINGS

In the drawings:

FIG. 1 is a perspective view of a catheter and medical tubing securement device in accordance with the invention;

FIG. 2 is an exploded perspective view of the securement device of FIG. 1;

FIG. 3 is a cross-sectional view taken along the line 3-3 in FIG. 1; and

FIG. 4 is a perspective view of an alternative embodiment of the invention.

### DETAILED DESCRIPTION OF THE INVENTION

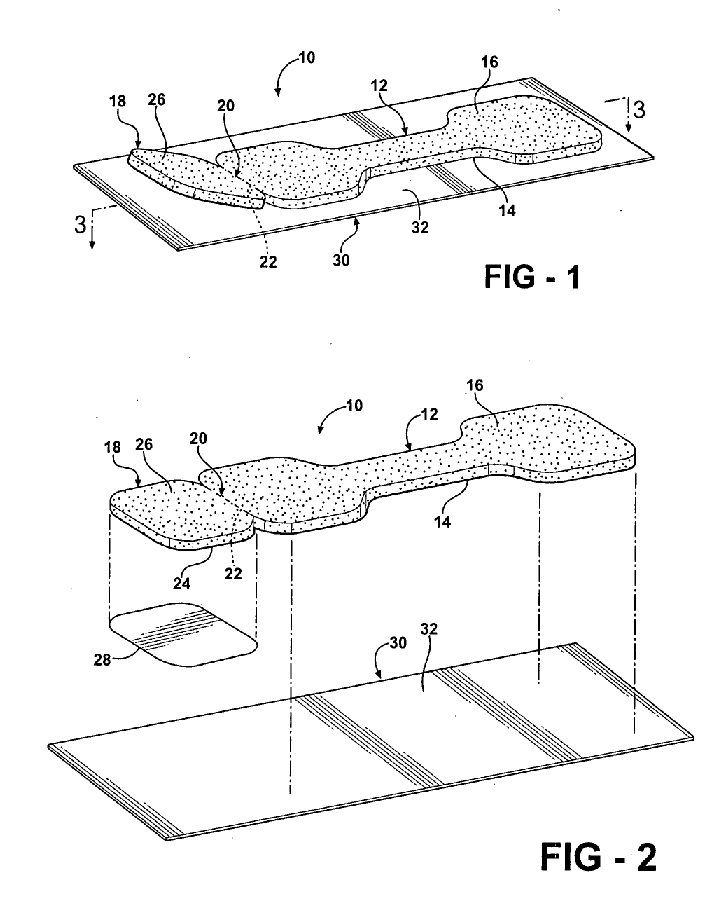

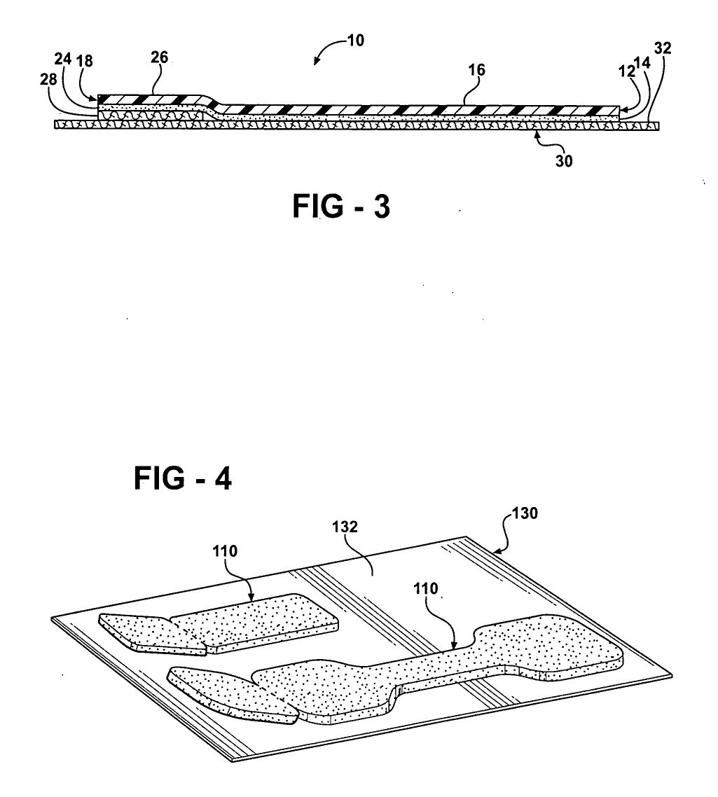

Referring now to the drawings in detail, numeral 10 generally indicates a catheter and medical tubing securement device in accordance with the invention. As illustrated in FIGS. 1 through 3, the securement device 10 includes a layer member 12 having an adhesive coated side 14 and an opposite non-adhesive side 16. The adhesive on the adhesive coated side 14 may be any suitable medical grade adhesive or similar. The adhesive coated side 14 is adherable to a patient's skin, as well as to medical tubing, catheter hubs, and other related fittings and connectors.

The securement device 10 further includes a tab 18 adjacent the layer member 12. The tab 18 generally defines a tab portion of the securement device 10, and the remainder of the securement device 10 is generally a securing portion. The tab 18 is removably connected to the layer member 12 along a perforated line 20 or similar. The perforated line 20 defines a boundary between the layer member 12 and the tab 18. The material between perforations of the perforated line 20 form frangible lands 22. The frangible lands 22 connect the tab 18 to the layer member 12 until a threshold tearing force is applied to frangible lands 22.

The tab 18 may have opposite non-adhesive sides. Alternatively, the tab 18 may have an adhesive coated side 24 and an opposite non-adhesive side 26. The adhesive coated side 24 of the tab 18 may correspond with the adhesive coated side 14 of the layer member 12. In other words, the tab adhesive coated side 24 and the layer member adhesive coated side 14 may both face generally the same direction. A release liner 28 or other paper material may be disposed on the adhesive coated side 24 of the tab 18. The release liner 28 may generally correspond in shape with the tab 18 such that the release liner 28 generally does not extend beyond an outer edge of the tab.

A second release liner 30 having a tackless side 32 may be removably disposed on the adhesive coated side 14 of the layer member 12. The tackless side 32 may include a layer of silicon material or similar. The release liner 30 may generally extend beyond an outer edge of the layer member 12 and the tab 18, although the release liner 30 may alternatively extend only to the outer edge of the layer member and tab. The release liner 30 protects the layer member 12 from becoming inadvertently stuck to an object prior to its use. If release liner 28 is disposed on the tab adhesive side 24, then release liner 28 is adjacent the tackless side 32 of the release liner 30 and may freely contact release liner 30. In an alternative arrangement, if release liner 28 is not present, the tab adhesive side 24 may releasably contact the tackless side 32 of release liner 30.

The layer member 12 and tab 18 may be formed from a foam material, an open/closed cell foam, a foam-like material, or another similarly suitable material. The layer member 12 may also be integral with the tab 18. For example, the layer member 12 may be formed from one piece of material.

The layer member 12 may have one of a numerous variety of shapes. For example, the layer member 12 may have an elongated rectangular shape or a bone shape. Alternatively, the layer member 12 may have a T-shape, a circular shape, or an X-shape. The tab 18 may also have one of a variety of shapes, although it is preferable that the tab 18 have a size and shape that can be grasped by a person's thumb and at least one finger. Therefore, a square, rectangular, or circular shape is preferred for the tab 18. Further, the tab 18 is usually smaller in size relative to the layer member 12.

In order to use the securement device 10, a user such as a health care provider may grip the securement device 10 by the tab 18. Since the tab 18 either lacks an adhesive coated side or has a release liner covering the adhesive, it is easy to pinch the tab 18 between a thumb and finger. Also, the thumb and finger do not stick to the tab 18, so the securement device 10 can be easily manipulated and positioned. Once the user has a grip on the tab 18, the user can gently pull on the tab to release the layer member 12 from the release liner 30. During this step, it may help for the user to use his/her other hand to hold the release liner 30.

With the adhesive coated side 14 of the layer member 12 exposed, the user may then position the layer member 12 about a patient and apply the layer member 12 to secure a catheter or medical tubing to the patient. Once the layer member 12 is adhered to the patient, the user may gently remove the tab 18 from the layer member 12 by tearing the frangible lands 22. It is desirable to remove the tab 18 after the layer member 12 is applied because this eliminates a point where pealing of the layer member 12 away from the patient's skin could begin. Removing the tab 18 also eliminates a surface that could abrade the patient's skin. The removed tab 18 may be discarded in an appropriate waste receptacle.

Turning to FIG. 4, more than one securement device 110 may be releasably mounted on a tackless side 132 of release liner 130. For example, two securement devices 110 may be separately mounted on the release liner 130 in a spaced relationship relative to each other. This allows for the added convenience of providing more than one shape of securement device 110 on a single carrier release liner 130.

Although the invention has been described by reference to specific embodiments, it should be understood that numerous changes may be made within the spirit and scope of the inventive concepts described. Accordingly, it is intended that the invention not be limited to the described embodiments, but that it have the full scope defined by the language of the following claims.

What is claimed is:

1. A catheter and medical tubing securement device comprising:
a layer member having an adhesive coated side and an opposite non-adhesive side, said adhesive coated side being adherable to a patient's skin; and
an adjacent tab being removably connected to said layer member;
wherein a user may manipulate said layer member by grasping said tab, and said tab may be removed from said layer member.

2. The catheter and medical tubing securement device of claim 1, including perforations between said layer member and adjacent tab, and wherein frangible lands between said perforations define a perforated boundary line.

3. The catheter and medical tubing securement device of claim 1, wherein said tab has opposite non-adhesive sides.

4. The catheter and medical tubing securement device of claim 1, wherein said tab has an adhesive coated side and an opposite non-adhesive side.

5. The catheter and medical tubing securement device of claim 4, wherein said adhesive coated side of said tab corresponds with said adhesive coated side of said layer member.

6. The catheter and medical tubing securement device of claim 4, including a release liner disposed on said adhesive coated side of said tab.

7. The catheter and medical tubing securement device of claim 6, wherein said release liner generally corresponds in shape with said tab.

8. The catheter and medical tubing securement device of claim 1, wherein said tab is integral with said layer member.

9. The catheter and medical tubing securement device of claim 1, including a release liner having a tackless side removably disposed on said layer member adhesive coated side.

10. The catheter and medical tubing securement device of claim 1, wherein said layer member comprises a foam material.

11. The catheter and medical tubing securement device of claim 1, wherein said tab comprises a foam material.

12. A catheter and medical tubing securement device comprising:
a layer member having an adhesive coated side and an opposite non-adhesive side, said adhesive coated side being adherable to a patient's skin;
said layer member including a tab portion and an adjacent securing portion; and
a boundary between said tab portion and said securing portion defined by perforations extending through said layer member;
wherein said layer member may be grasped by said tab portion, and said tab portion may be removed from said securing portion by tearing said tab portion from said securing portion along said boundary.

13. The catheter and medical tubing securement device of claim 12, including a first release liner removably disposed on said layer member adhesive coated side.

14. The catheter and medical tubing securement device of claim 13, wherein said release liner generally extends beyond an outer edge of said layer member.

15. The catheter and medical tubing securement device of claim 13, including a second release liner removably disposed on said adhesive coated side of said tab portion of said layer member intermediate said tab portion and said first release liner.

16. The catheter and medical tubing securement device of claim 12, wherein said tab portion is free of adhesive.

17. The catheter and medical tubing securement device of claim 12, wherein said layer member comprises a foam material.
